(19) United States
(12) Patent Application Publication
(10) Pub. No.: US 2003/0088304 A1
(43) Pub. Date: May 8, 2003

(54) Stent holder

(75) Inventors: Irene Rueeger, Bachs (CH); Silvio Schaffner, Berlingen (CH); Suk-Woo Ha, Langwiesen (CH)

Correspondence Address: SHINJYU GLOBAL IP COUNSELORS, LLP, 1233 20TH STREET, NW, SUITE 700, WASHINGTON, DC, 20036-2680, US

(73) Assignee: JOMED NV., NS Ulestraten (NL)

(21) Appl. No.: 10/265,197

(22) Filed: Oct. 7, 2002

(30) Foreign Application Priority Data
Oct. 8, 2001 (EP) 01123996.9

(51) Int. Cl. A61F 2/06

(52) U.S. Cl. 623/1.12; 623/1.2; 623/1.22

(57) ABSTRACT

A stent holder is configured for holding a self-expandable stent for insertion into a body vessel. In order to prevent the stent from migrating on a holder body of the stent holder, said holder body comprises two fixing or fixation elements for temporarily fastening opposite stent ends of stent.

### BACKGROUND OF THE INVENTION

1. Field of the Invention

The present invention generally relates to a stent holder for self-expandable stents.

2. Background Information

Stent holders typically have a holder body upon which the stent is temporarily disposed in its contracted state. An outer sheath is shifted over the stent in order to hold the stent in its contracted position until the stent has been moved to the site of implantation. In order to implant the stent, the outer sheath is retracted so that the stent can self-expand into its supporting position, e.g., within the stenosis of a body vessel.

Especially when the stent on the stent holder is pushed forward to the curved vessels, it tends to move or migrate on the holder body as it is not positively fixed thereon. So, high stresses may be created in the stent so that the stent-catheter unit may be stuck in the vessel. Additionally, advancement through tortuous anatomy may longitudinally compress the stent, thereby significantly increasing the force required to release the stent. Unwanted compression may also occur during crimping or mounting.

In view of the above, it will be apparent to those skilled in the art from this disclosure that there exists a need for an improved stent holder. This invention addresses this need in the art as well as other needs, which will become apparent to those skilled in the art from this disclosure.

### SUMMARY OF THE INVENTION

It has been discovered that the above mentioned problems can be overcome by providing fixing means which can be formed by two enlargements on the holder body. The positioning of the fixing means can be adapted to the length of the respective stent to be mounted on the holder body of the stent holder.

Preferably, the enlargements can be integral parts of the holder body. However, an alternative are enlargements that are formed by glue layers to be applied to the holder body. With this embodiment, the positioning of the fixing means can easily be adapted to different stent lengths.

It is therefore an object of the present invention to provide a stent holder for a self-expandable stent in which the stent holder is able to prevent the stent from moving or migrating when it is moved through body vessels, especially curved body vessels.

The forgoing object can basically be achieved by providing a stent holder for holding a stent in which the stent holder comprises an elongated holder body having at least one fixation element for temporarily securing the stent thereon.

As the stent holder according to the present invention enables a fixation of the stent on at least one fixation element of the holder body, the stent body between its ends is flexibly supported on the holder body, and thus can easily follow a movement of the holder body through curved body vessels without a tendency to move or migrate.

Thus, the stent holder of the present invention is especially advantageous for long stents.

The fixation of the stent on the fixation element or the fixing means can, for example, be achieved by frictional forces or geometric constraint.

In accordance with one aspect of the present invention, the holder body of the stent holder has at least two fixation elements spaced apart from one another. Preferably, the two fixation elements are spaced apart from one another by a distance in the range of about 1 cm to about 10 cm.

In accordance with another aspect of the present invention, the fixation element comprises at least one enlargement of the holder body. Preferably, the enlargement is an integral part of the holder body. More preferably, the enlargement is formed from an enlargement chosen from the group consisting of glue layers, shrink tubing, an attachment to the holder body, an integral portions of the holder body, and combinations thereof.

These and other objects, features, aspects and advantages of the present invention will become apparent to those skilled in the art from the following detailed description, which, taken in conjunction with the annexed drawings, discloses preferred embodiments of the present invention.

### BRIEF DESCRIPTION OF THE DRAWINGS

Referring now to the attached drawings which form a part of this original disclosure:

FIG. 1 is a schematically simplified side view of a stent holder in accordance with one embodiment of the present invention;

FIG. 2 is a schematically simplified side view of the stent holder body of the stent holder of FIG. 1, prior to forming a pair of fixation elements;

FIG. 3 is an enlarged partial side view, corresponding to a part of FIG. 2, of a portion of the stent holder body to depict a first step of creation of a fixing means;

FIG. 4 is an enlarged partial side view, corresponding to FIG. 3, of a portion of the stent holder body to show the fixing means formed of glue (layers of glue);

FIG. 5 is an enlarged partial side view, corresponding to FIG. 3, of a portion of the stent holder body to show the fixing means formed integrally with the stent holder body; and

FIG. 6 is an enlarged partial side view, corresponding to FIG. 3, of a portion of the stent holder body to show the fixing means formed of shrink tubing over an annular fixing member (an attachment).

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

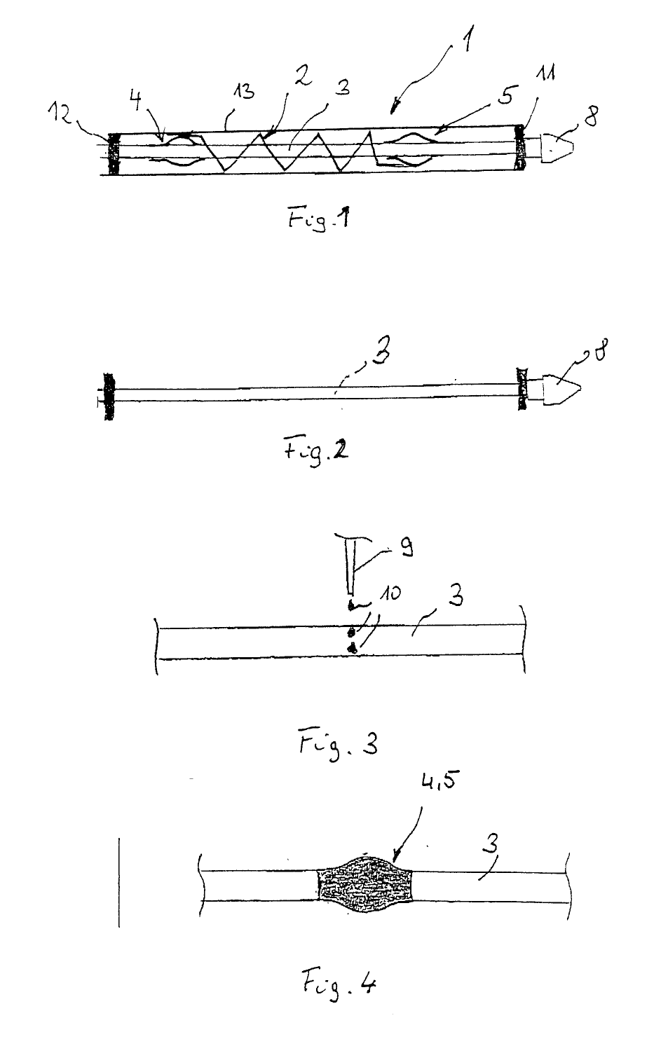

Referring initially to FIG. 1, a stent holder 1 is illustrated in accordance with a first embodiment of the present invention.

FIG. 1 shows an embodiment of the stent holder 1 of the present invention for holding a self-expandable stent 2 during insertion into a body vessel. The stent 2 and the stent holder 1 form a stent delivery system or assembly for implanting the stent 2 into a body vessel. The stent 2 is a flexible, generally cylindrical, expandable, coiled stent having a discrete proximal end section, a distal end sections and a center passage therebetween. The stent 2 is wound circumferentially around the stent holder 1. Thus, the stent 2 is formed from coiled material in a conventional manner.

The stent holder 1 comprises an elongated stent holder body 3, preferably in the form of a circular cylindrical rod that is disposed in the center passage of the stent 2. The stent holder body 3 includes a body tip 8, two end supporting means or elements 11 and 12 and an outer sheath 13. The outer sheath 13 is pushed over the stent 2 in its contracted position in order to prevent the stent 2 from expanding when mounted on the holder body 3.

FIG. 1 furthermore shows two fixation elements 4 and 5 that form fixing means. The fixation elements 4 and 5 are disposed on the holder body 3, and are axially spaced apart from each other on the holder body 3. The distance between the fixation elements 4 and 5 depends on the axial length of the stent 2 such that the fixation elements 4 and 5 can temporarily hold the stent 2 on the holder body 3. An optimum spacing of the fixation elements 4 and 5 is a range between about 1 cm to about 10 cm for most stents.

Because of this fixation of the stent 2 on the holder body 3, the stent 2 can be prevented from moving or migrating during a movement of the stent holder 1, particularly through curved body vessels. Additionally, longitudinal compression of the stent 2 may be reduced during mounting or deployment, thereby reducing required mounting or delivery forces.

FIGS. 2 and 3 show one exemplary technique for forming the fixation elements 4 and 5, seen in FIG. 4, from glue layers on the holder body 3. In FIG. 3, a tip 9 of a glueing device is shown that continuously drops glue 10 onto the surface of the holder body 3.

As per completing the form of the glue layer, as shown in FIG. 4, same could, for example, be heat-treated, UV cured, etc., in order to harden the glue spots.

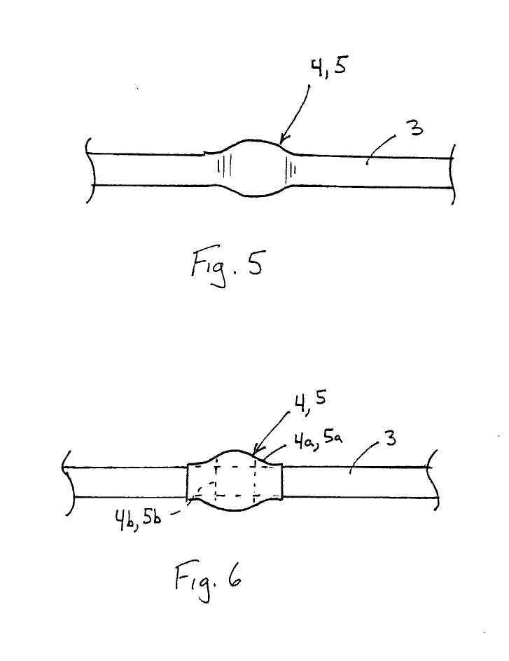

As will be apparent to those of skill in the art, any number of fixation elements may be provided. Additionally, the spacing and/or size of the elements may be varied, as needed. Furthermore, additional techniques for forming fixation elements will be apparent, in addition to glue layers. For example, the fixation elements 4 and 5 could be formed integrally with the holder body 3 as seen in FIG. 5. Alternatively, the fixation elements 4 and 5 could be formed from shrink tubing 4a, 5a over an annular fixing member 4b, 5b (an attachment) as seen in FIG. 6. Of course, the fixation elements 4 and 5 could be attached to the holder body 3 in other ways. The appended claims are intended to cover all such changes or modifications that fall within the true spirit and scope of the present invention.

The terms of degree such as “substantially”, “about” and “approximately” as used herein mean a reasonable amount of deviation of the modified term such that the end result is not significantly changed. For example, these terms can be construed as including a deviation of at least ±5% of the modified term if this deviation would not negate the meaning of the word it modifies.

This patent application claims priority to European Patent Application No. 01 123 996.9. The entire disclosure of European Patent Application No. 01 123 996.9 is hereby incorporated herein by reference.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents. Thus, the scope of the invention is not limited to the disclosed embodiments.

What is claimed is:

1. A stent holder for holding a stent, the stent holder comprising:
an elongated holder body having at least one fixation element for temporarily securing the stent thereon.

2. The stent holder as set forth in claim 1, wherein
the at least one fixation element comprises at least one enlargement of the holder body.

3. The stent holder as set forth in claim 2, wherein
the at least one enlargement is an integral part of the holder body.

4. The stent holder as set forth in claim 2, wherein
the at least one enlargement is formed from an enlargement chosen from the group consisting of glue layers, shrink tubing, an attachment to holder body, an integral portion of holder body, and combinations thereof.

5. The stent holder as set forth in claim 1, wherein
the holder body has at least two fixation elements spaced apart from one another.

6. The stent holder as set forth in claim 5, wherein
the at least two fixation elements are spaced apart from one another by a distance in the range of about 1 cm to about 10 cm.

7. The stent holder as set forth in claim 5, wherein
the at least two fixation elements are an integral part of the holder body.

8. The stent holder as set forth in claim 5, wherein
the at least two fixation elements are formed from an enlargement chosen from the group consisting of glue layers, shrink tubing, an attachment to the holder body, an integral portions of the holder body, and combinations thereof.

9. A stent assembly comprising:
a stent having a central passage; and
a stent holder comprising an elongated holder body having at least one fixation element for temporarily securing the stent thereon.

10. The stent assembly as set forth in claim 9, wherein
the at least one fixation element comprises at least one enlargement of the holder body.

11. The stent assembly as set forth in claim 10, wherein
the at least one enlargement is an integral part of the holder body.

12. The stent assembly as set forth in claim 10, wherein
the at least one enlargement is formed from an enlargement chosen from the group consisting of glue layers, shrink tubing, an attachment to holder body, an integral portion of holder body, and combinations thereof.

13. The stent assembly as set forth in claim 9, wherein
the holder body has at least two fixation elements spaced apart from one another.

14. The stent assembly as set forth in claim 13, wherein
the at least two fixation elements are spaced apart from one another by a distance in the range of about 1 cm to about 10 cm.

15. The stent assembly as set forth in claim 13, wherein
the at least two fixation elements are an integral part of the holder body.

16. The stent assembly as set forth in claim 13, wherein
the at least two fixation elements are formed from an enlargement chosen from the group consisting of glue layers, shrink tubing, an attachment to the holder body, an integral portions of the holder body, and combinations thereof.
